(19) United States
(12) Patent Application Publication
(10) Pub. No.: US 2010/0217532 A1
(43) Pub. Date: Aug. 26, 2010

(54) SYSTEMS AND METHODS FOR IDENTIFYING STRUCTURALLY OR FUNCTIONALLY SIGNIFICANT AMINO ACID SEQUENCES

(75) Inventors: Adam G. Marsh, Lewes, DE (US); Joseph J. Grzymski, Reno, NV (US)

Correspondence Address: RATNERPRESTIA, P.O. BOX 1596, WILMINGTON, DE, 19899, US

(73) Assignees: University of Delaware, Newark, DE (US); Board of Regents of the Nevada System of Higher Education, on Behalf of the Desert Research Instit, Reno, NV (US)

(21) Appl. No.: 12/546,285

(22) Filed: Aug. 24, 2009

Related U.S. Application Data
(60) Provisional application No. 61/208,513, filed on Feb. 25, 2009.

(51) Int. Cl. G06F 19/00; G01N 33/48

(52) U.S. Cl. 702/19

(57) ABSTRACT

Methods and computer readable storage mediums for identifying structurally or functionally significant amino acid sequences encoded by a genome are disclosed. At least one structurally or functionally significant amino acid sequence encoded by a genome may be identified by compiling an observed frequency for each of a plurality of amino acid words encoded by the genome, calculating with a computer an expected frequency for each of the plurality of amino acid words encoded by the genome, and identifying at least one structurally or functionally significant amino acid sequence encoded by the genome based at least in part on the observed and expected frequencies for each of the plurality of amino acid words encoded by the genome.

### CROSS REFERENCE TO RELATED APPLICATIONS

This application is related to and claims the benefit of U.S. Provisional Application No. 61/208,513 entitled Systems and Methods for Identifying Structurally or Functionally Significant Amino Acid Sequences filed on Feb. 25, 2009, the contents of which are incorporated fully herein by reference.

### FIELD OF THE INVENTION

The present invention relates to the field of drug development, and more particularly to systems and methods for identifying structurally or functionally significant amino acid sequences.

### BACKGROUND OF THE INVENTION

Pathogenic bacteria are bacteria which may infect a host organism and thereby cause disease or illness. Infection with pathogenic bacteria may be treated with antibiotics drugs designed to target and kill certain pathogenic bacteria. Recent years have seen an increasing number of antibiotic-resistance pathogenic bacterial strains appear in the public domain. In this same time frame, the introduction of new antibiotic drugs has declined. Therefore, there is a need for new antibiotic drugs to target the increasing number of pathogenic bacteria, and consequently a need for new research strategies for developing such drugs.

### SUMMARY OF THE INVENTION

Aspects of the present invention are embodied in systems, methods, and computer readable storage mediums for identifying structurally or functionally significant amino acid sequences encoded by a genome. At least one structurally or functionally significant amino acid sequence encoded by a genome may be identified by compiling an observed frequency for each of a plurality of amino acid words encoded by the genome, calculating with a computer an expected frequency for each of the plurality of amino acid words encoded by the genome, and identifying at least one structurally or functionally significant amino acid sequence encoded by the genome based at least in part on the observed and expected frequencies for each of the plurality of amino acid words encoded by the genome.

In accordance with another aspect of the present invention, a structurally or functionally significant amino acid sequence in the protein of a pathogen may be targeted by compiling an observed frequency for each of a plurality of amino acid words encoded by the genome of the pathogen, calculating with a computer an expected frequency for each of the plurality of amino acid words encoded by the genome of the pathogen, identifying at least one structurally or functionally significant amino acid sequence encoded by the genome of the pathogen based at least in part on the observed and expected frequencies for each of the plurality of amino acid word encoded by the genome of the pathogen, and developing a drug configured to interact with the at least one structurally or functionally significant amino acid sequence encoded by the genome of the pathogen.

### BRIEF DESCRIPTION OF THE DRAWINGS

The invention is best understood from the following detailed description when read in connection with the accompanying drawings. Included in the drawings are the following figures:

FIG. 1 is a block diagram depicting an exemplary system for identifying significant amino acid sequences encoded by a genome in accordance with one aspect of the present invention;

FIG. 2 is a flow chart of exemplary steps providing an overview for identifying significant amino acid sequences encoded by a genome for use in developing antibiotic drugs in accordance with an aspect of the present invention;

FIG. 3 is a flow chart of exemplary steps for identifying significant amino acid sequences encoded by a genome in accordance with an aspect of the present;

FIG. 4 is a flow chart of exemplary steps for outputting genome word dictionaries in accordance with an aspect of the present invention;

FIG. 5 is an example for determining a selection score of an amino acid sequence in accordance with an aspect of the present invention;

FIG. 6A is an exemplary graph depicting the residual distance between an observed and expected word count for a genome in accordance with an aspect of the present invention;

FIG. 6B is another exemplary graph depicting the residual distance between an observed and expected word count for a genome in accordance with an aspect of the present invention; and

FIG. 7 is an exemplary chart depicting a selection score for amino acid sequences encoded by a genome in accordance with an aspect of the present invention.

### DETAILED DESCRIPTION OF THE INVENTION

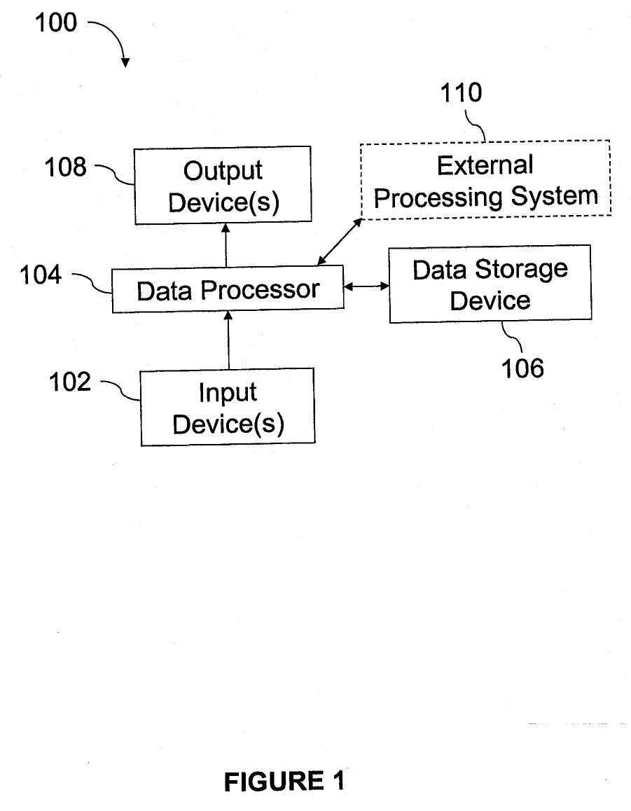

FIG. 1 depicts an exemplary system 100 for identifying structurally or functionally significant amino acid sequences encoded by the nucleic acid sequences from an organism's genome in accordance with one aspect of the present invention. The genome may be from a human pathogen such as a bacterium. The structurally or functionally significant amino acid sequences may represent functional sites on the bacterial proteins that may be vulnerable to antibiotic drug targeting. The pathogenic bacteria targeted may include any bacterial pathogens, including for example the following species: Clostridium difficile str. 630, Shigella dystenteriae, Helicobacter pylori str. HPAG1, Corynebacterium diphtheriae, Neisseria meningitides str. FAM18, and Ricksettsia typhi str. Wilmington.

As used herein, a genome for bacteria refers to the complete genetic sequence of the bacteria. Each genome includes multiple genes that encode various polypeptide sequences. Some of the polypeptide sequences encoded by the genome include protein sequences. Each protein sequence encoded by the genome is comprised of a sequence of amino acids.

As a general overview, system 100 includes one or more input device(s) 102, a data processor 104, a data storage device 106, and one or more output device(s) 108. System 100 may optionally include an external processing system 110. Additional details of system 100 are provided below.

Input device(s) 102 is/are coupled to data processor 104 and may be used to provide electronic data from a user or electronic device to data processor 104. In one exemplary embodiment, the electronic data may include data relating to one or more genomes. In another exemplary embodiment, the electronic data may include the observed frequency of each amino acid word in the protein sequences encoded by the is genome. Additionally, an input device 102 may be used to provide user instructions to data processor 104. Input device(s) 102 may include a server, database, keyboard and/or other computer peripheral devices capable of providing electronic data to a data processor.

Data processor 104 receives electronic data from input device 102 and processes the electronic data. Data processor 104 may store received electronic data or processed electronic data in data storage device 106 (described below). In one exemplary embodiment, data processor 104 receives electronic data including data relating to one or more genomes. In another exemplary embodiment, data processor 104 receives electronic data including an observed frequency of each amino acid word in the protein sequences encoded by a genome.

Data processor 104 is configured to process electronic data. Data processor 104 may transform the electronic data into another format. In one exemplary embodiment, the transformed electronic data may include an amino acid word dictionary for a genome. In another exemplary embodiment, the transformed electronic data may include one or more selection scores (described below) for a genome. The transformed electronic data may be stored in data storage device 106 (described below), or transmitted to output device 108 (described below).

Data storage device 106 stores electronic data received from data processor 104. In one exemplary embodiment, data processor 104 may store electronic data including data relating to one or more genomes on data storage device 106. In another exemplary embodiment, data processor 104 may store electronic data including one or more amino acid word dictionaries for one or more genomes on data storage device 106. In yet another exemplary embodiment, data processor 104 may store electronic data including one or more selection scores for one or more genomes on data storage device 106. Data processor 104 may access the electronic data stored on data storage device 106. A suitable data storage device for use with the present invention will be understood by one of skill in the art from the description herein.

An exemplary system including suitable processors and data storage devices for use with the present invention includes a Sun Microsystems SunFire V60x cluster, featuring 128 dual processor 2.8 GHx Xeon CPUs, 7 quad-processor Sunfire X4100M2 nodes, a 48 node Myrinet Switch, 160 GB of memory, and over a terabyte of disk storage. Other suitable data processors and data storage devices will be understood by one skilled in the art from the description herein.

Output device(s) 108 is/are coupled to data processor 104 and may be used to present electronic data received from data processor 104 to a user. In one exemplary embodiment, the electronic data may include one or more amino acid word dictionaries for one or more genomes. In another exemplary embodiment, the electronic data may include one or more selection scores for one or more genomes. Output device(s) 108 may include a computer display, printer, or other computer peripheral device capable of generating output to a user from received electronic data.

An optional external processing system 110 is configured to exchange electronic data with data processor 104 and may perform one or more of the functions performed by data processor 104. Additionally, external processing system 110 may provide electronic data to data processor 104 for further processing. A suitable external processing system for use with the present invention will be understood by one skilled in the art from the description herein.

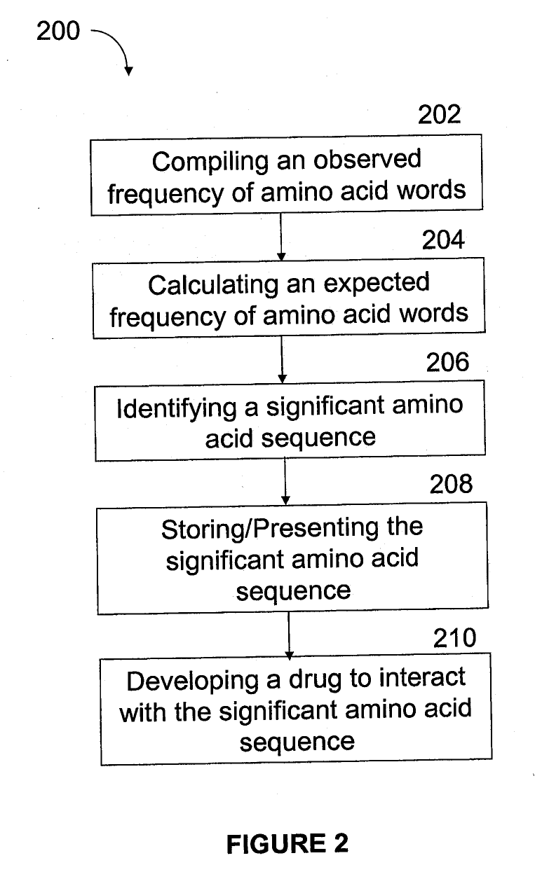

FIG. 2 is a flow chart 200 of exemplary steps for identifying significant amino acid sequences in the protein sequences encoded by a genome for bacteria for use in developing antibiotic drugs in accordance with an aspect of the present invention. To facilitate description, the steps of FIG. 2 are described with reference to the system components of FIG. 1. As referenced herein, any step employing data processor 104 may substitute external processing system 110 to perform all or part of the necessary processing function. It will be understood by one of skill in the art from the description herein that one or more steps may be omitted and/or different components may be utilized without departing from the scope of the present invention.

In step 202, an observed frequency of amino acid words in the protein sequences encoded by a genome is compiled. In an exemplary embodiment, data processor 104 receives data relating to a genome from input device(s) 102. Data processor 104 may then count the number of times each amino acid word occurs in each protein sequence encoded by the genome, and compile a list of the observed frequencies for each amino acid word. The list of the observed frequencies of amino acid words may be stored in data storage device 106.

In step 204, an expected frequency of amino acid words in each protein sequence encoded by a genome is calculated, e.g., with a general or specific purpose computer. The expected frequency of each amino acid word may be calculated based at least in part on the observed amino acid word frequency list compiled in step 202. In an exemplary embodiment, data processor 104 calculates an expected frequency of an amino acid word based on the observed frequencies of two or more amino acid subwords that make up the amino acid word. As used herein, an amino acid subword is an amino acid word occurring within another amino acid word. Data processor 104 10 may then compile a list of the expected frequencies for each amino acid word. The list of the expected frequencies of amino acid words may then be stored in data storage device 106.

In step 206, a structurally or functionally significant amino acid sequence is identified. The structurally or functionally significant amino acid sequence may be identified based at least in part on the observed and expected amino acid word frequencies compiled in steps 202 and 204. In an exemplary embodiment, data processor 104 generates a selection score for each amino acid sequence in each protein sequence encoded by the genome based on the difference between the expected and observed word frequencies for each amino acid in the sequence. The maximum selection scores correspond to amino acid sequences occurring more frequently in all of the protein sequences encoded the genome than is expected from its expected frequency, which indicates that it is structurally or functionally significant to the bacteria.

The identification of the structurally or functionally significant amino acid sequence may be additionally based on a comparison of the amino acid word frequencies in the protein sequences encoded by the genome (e.g., a genome of a pathogenic bacteria) to the amino acid word frequencies in protein sequences encoded by a related genome (e.g., a genome of a non-pathogenic bacteria related to the pathogenic bacteria). In accordance with this embodiment, differences between the amino acid frequencies of the pathogenic genome and the non-pathogenic genome may be used to identify amino acid words that are significant to the pathogenic bacteria but not to the non-pathogenic bacteria, e.g., amino acid words having a greater frequency in the pathogenic bacteria than the non-pathogenic bacteria. This may provide further information on the different effects of natural selection on the genome of a pathogen as opposed to the effects of natural selection on the genome of a non-pathogen.

In step 208, the structurally or functionally significant amino acid sequence is stored and/or presented. In one exemplary embodiment, the selection scores for one or more structurally or functionally significant amino acid sequences may be stored in data storage device 106. In another exemplary embodiment, data processor 104 may transmit electronic data to output device(s) 108. The electronic data may include the selection scores for one or more structurally or functionally significant amino acid sequences in the genome. Output device(s) 108 may then present the selection scores to a user by, for example, a chart or graph indicating the comparative height of the selection scores for the one or more structurally or functionally significant amino acid sequences presented on a monitor or printed on paper. Electronic data transmitted to output device(s) 108 may be at least temporarily stored, e.g., in a video buffer (not shown).

Identifying one or more structurally or functionally significant amino acid sequences of a pathogen may be useful for designing drugs to target structurally or functionally significant parts of the pathogen. However, identifying structurally or functionally significant amino acid sequences may have other uses. Such uses may include identifying patterns of gene structure and organization, identifying critical genes/pathways in a pathogen, identifying latent pathogen genes in environmental genomes, identifying potential new or emergent pathogen diseases, or identifying patterns of emergent pathogen evolution. It will be understood by one skilled in the art that in these applications, the following step 210 may be omitted.

In step 210, an antibiotic drug is developed to interact with the structurally or functionally significant amino acid sequence. The antibiotic drug may be configured to target one or more structurally or functionally significant amino acid sequences of a pathogen. In an exemplary embodiment, an antibiotic drug is designed to target an amino acid sequence having a high selection score in a pathogen. In a further exemplary embodiment, an antibiotic drug is designed to target an amino acid sequence having a high selection score in multiple pathogens, to increase the effectiveness of the drug. The development of a drug to target a selected amino acid sequence will be known to one of skill in the art.

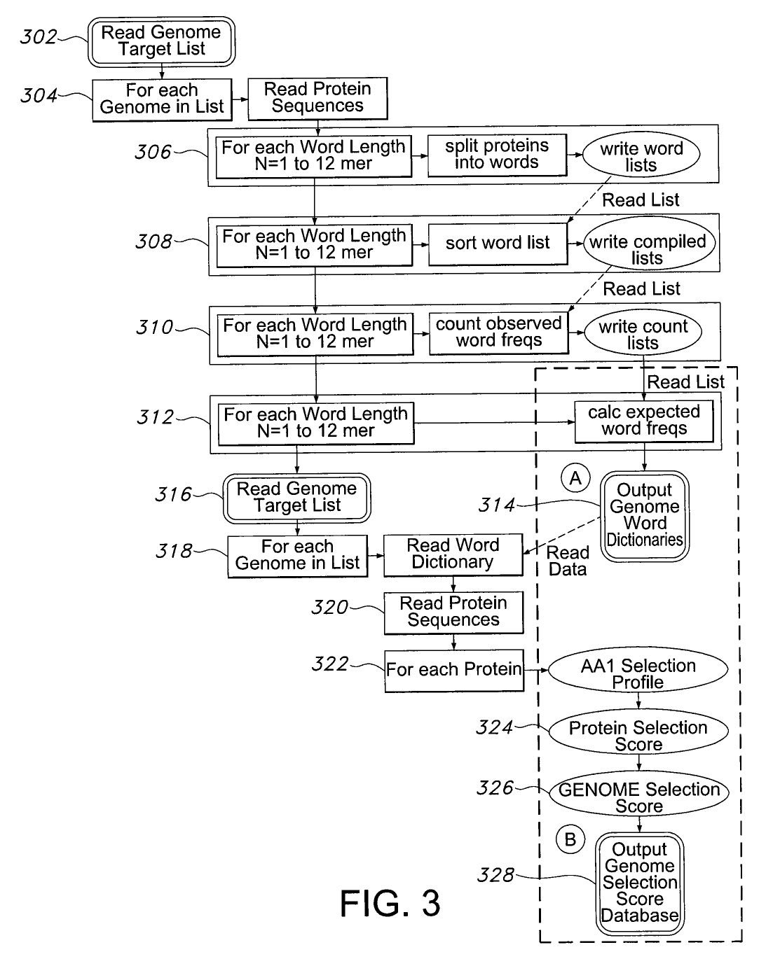

FIG. 3 is a flow chart 300 of exemplary steps for identifying significant amino acid sequences in the protein sequences encoded by a genome in accordance with an aspect of the present invention. To facilitate description, the steps of FIG. 3 are described with reference to the system components of FIG. 1. As referenced herein, any step employing data processor 104 may substitute external processing system 110 to perform all or part of the necessary processing function. It will be understood by one of skill in the art from the description herein that one or more steps may be omitted and/or different components may be utilized without departing from the spirit and scope of the present invention.

In step 302, a genome target list is read. In an exemplary embodiment, data processor 104 receives a genome target list from input device(s) 102. The genome target list may include one or more genomes identified by a user for which amino acid word dictionaries are desired to be created. For example, a user doing research on human pathogenic bacteria may identify particularly virulent pathogens for inclusion in the genome target list.

In step 304, the protein sequences in each genome on the genome target list are read. As noted above, each genome encodes multiple polypeptide sequences, of which a number of sequences are protein sequences. In an exemplary embodiment, data processor 104 may read a genome to determine what protein sequences it encodes in order to separately analyze each protein sequence.

In step 306, word lists are written for each protein sequence. In an exemplary embodiment, data processor 104 splits each protein sequence into amino acid words having a length of between one and twelve amino acids, although other lengths are contemplated. For example, the invention has been applied to pathogens having relatively large genomes such as eukaryotic pathogens (e.g., protozoa like Trypansoma (Chagas disease) and Plasmodia (malaria)). For these large genomes, the amino acid word dictionaries can be extended to 24 amino acids or more, while having enough depth to provide relevant information. Data processor 104 may write a list containing each amino acid word occurring in the protein sequence, e.g., to data storage device 106.

In step 308, the list of the words occurring in each protein sequence is compiled. In an exemplary embodiment, data processor 104 may compile the list of each amino acid word occurring more than once in the protein sequences encoded by a genome. The compiled amino acid word list may be stored in data storage device 106.

In step 310, the observed frequency of each amino acid word in the protein sequence is counted and written to a count list. In an exemplary embodiment, data processor 104 may count the observed occurrences of each amino acid word in the compiled list. Data processor 104 may calculate the frequency of each amino acid word in each protein sequence encoded by the genome by dividing the observed number of occurrences for each amino acid word by the number of amino acids in the protein sequence or genome. Data processor 104 may then write a list including the frequency for each amino acid word in the protein sequences. The list containing the observed amino acid word frequency may be stored in data storage device 106.

In step 312, the expected frequency of each amino acid word in each protein sequence is calculated. In an exemplary embodiment, the expected frequency of each amino acid word in a protein sequence may be derived from the probability of each amino acid word in the protein sequence occurring. Data processor 104 may calculate the probability of an amino acid word based on the probability of the occurrence of two or more amino acid subwords making up the amino acid word.

An exemplary algorithm for determining the probability of the occurrence of an amino acid word in the protein sequence may involve calculating the probability from the observed frequency of each amino acid word in the protein sequence. The probability of a 1-long amino acid word (i.e. a single amino acid) occurring within the protein sequence is equal to the frequency of the amino acid, i.e. the number of occurrences of that amino acid in a protein divided by the total number of amino acids in the protein. For example, if the amino acid “A” (for alanine) occurs 11 times in a protein of 100 amino acids, then the probability of the 1-long amino acid word p(A) is 11%. For a 2-long amino acid word, the probability may be determined to be one half of the probability of the first 1-long amino acid subword multiplied by the probability of the second 1-long amino acid subword. For example, if p(A) is 11%, and p(L) (for the 1-long amino acid word for leucine “L”) is 8%, then p(AL) (for the 2-long amino acid word “AL”) would be equal to one half of 0.11*0.08, or 0.44% (with the same probability existing for p(LA)). For N-long amino acid words (where N>2), the probability may be determined based on the probability of a 1-long amino acid subword and a (N−1)-long amino acid subword. For example, the probability of the amino acid word “VALK” occurring may be equal to the average of p(VAL)*p(K) and p(V)*p(ALK).

Using this algorithm, data processor 104 may calculate the probability of any amino acid word occurring based on the probability of two or more subwords of the amino acid word, which may be obtained using the list of observed frequencies of amino acid words in each protein. Data processor 104 may calculate the expected frequency of an amino acid word in a protein by multiplying the probability of the amino acid word occurring with the total number of amino acids in the protein. The expected amino acid word frequency for each amino acid word in each protein sequence encoded by the genome may be stored in data storage device 106.

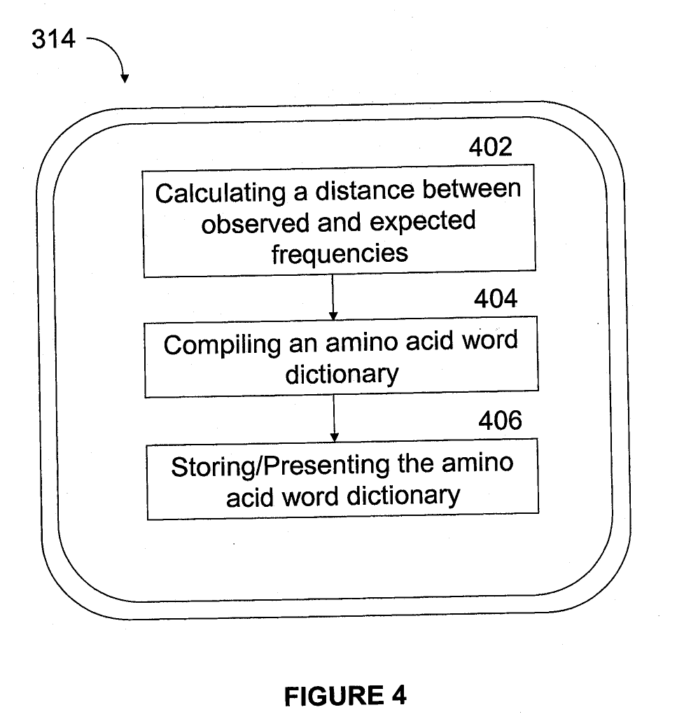

In step 314, a genome word dictionary is output, e.g., stored to data storage device 106 and/or transmitted to output device 108. In an exemplary embodiment, data processor 104 generates an amino acid word dictionary for each genome. The amino acid word dictionary may contain an entry for each amino acid word in each protein sequence encoded by the genome. Each entry for the amino acid word may include the word's observed frequency, expected frequency, and/or the difference between the observed and expected frequencies. After generating the amino acid word dictionary for each genome, data processor 104 may then store the amino acid word dictionary on data storage device 106 for later access. Additionally, data processor 104 may transmit electronic data including amino acid word dictionaries for each amino acid word in the genome to output device(s) 108. Output device(s) 108 may then present the amino acid word dictionaries to a user via a chart or graph, for example. FIG. 4, described below, depicts a flow chart of exemplary steps for performing step 314.

In step 316, a genome target list is read. Data processor 104 may receive the genome target list from input device(s) 102. The genome target list may be generated by a user. In an exemplary embodiment, the genome target list may be the same list of genomes read in step 302. In an alternative exemplary embodiment, the genome target list may be a list including genomes for which amino acid word dictionaries have been created, as described above in steps 304-314.

In step 318, the amino acid word dictionaries for each genome on the genome target list are read. In an exemplary embodiment, data processor 104 accesses amino acid word dictionaries stored by data storage device 106. Data processor 104 then reads the amino acid word dictionaries for each genome on the genome target list.

In step 320, the protein sequences for each genome in the genome target list are read. In an exemplary embodiment, data processor 104 may read each genome on the genome target list to determine what proteins sequences it encodes in order to separately analyze each protein sequence.

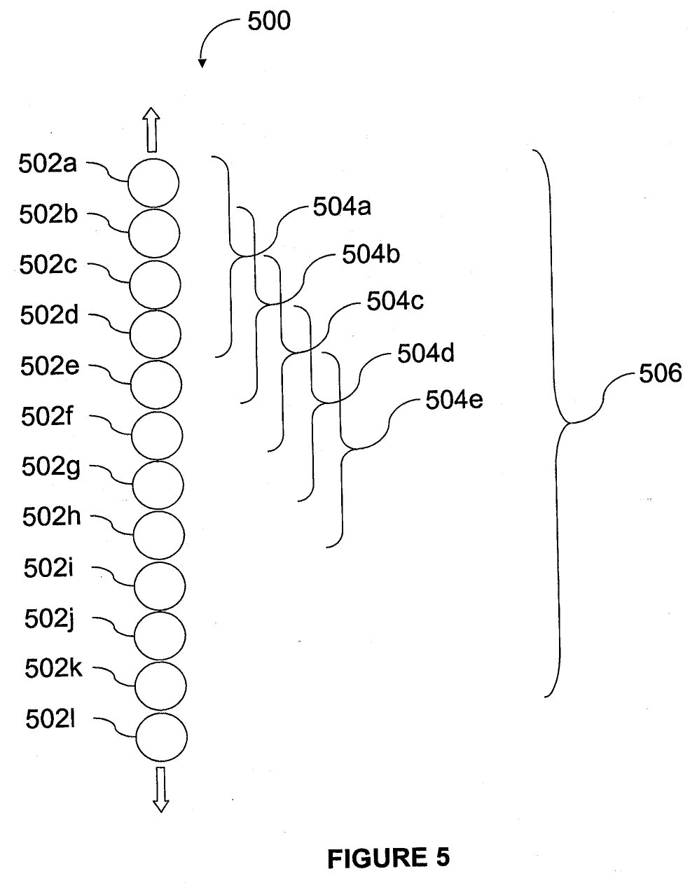

In step 322, an amino acid sequence selection score is determined for the amino acid sequences in each protein sequence. In an exemplary embodiment, data processor 104 calculates an amino acid sequence selection score based on the amino acid word dictionaries for each amino acid word in the protein sequence. Data processor 104 may assign an amino acid selection score to each amino acid occurring in the protein sequence. The amino acid selection score may be calculated by summing the distances between the observed and expected frequencies for each 4-long, 5-long, and 6-long word containing the amino acid. Data processor 104 may then examine all 13-long amino acid sequences in each protein. Data processor 104 may determine an amino acid sequence selection score for each 13-long amino acid sequence in each protein sequence encoded by the genome by summing the amino acid selection scores for each amino acid contained in the amino acid sequence. The amino acid sequence selection score may be stored in data storage device 106. FIG. 5, described below, depicts an exemplary amino acid sequence for further explaining the determination of a selection score in step 322.

In step 324, a protein selection score is determined. In an exemplary embodiment, data processor 104 may calculate a protein selection score for each protein encoded by a genome by summing the amino acid sequence selection scores for each 13-long amino acid sequence in the protein. The protein selection score may be stored in data storage device 106.

In step 326, a genome selection score is determined. In an exemplary embodiment, data processor 104 may calculate a genome selection score for the genome by summing the protein selection scores for each protein sequence encoded by the genome. The genome selection score may be stored in data storage device 106.

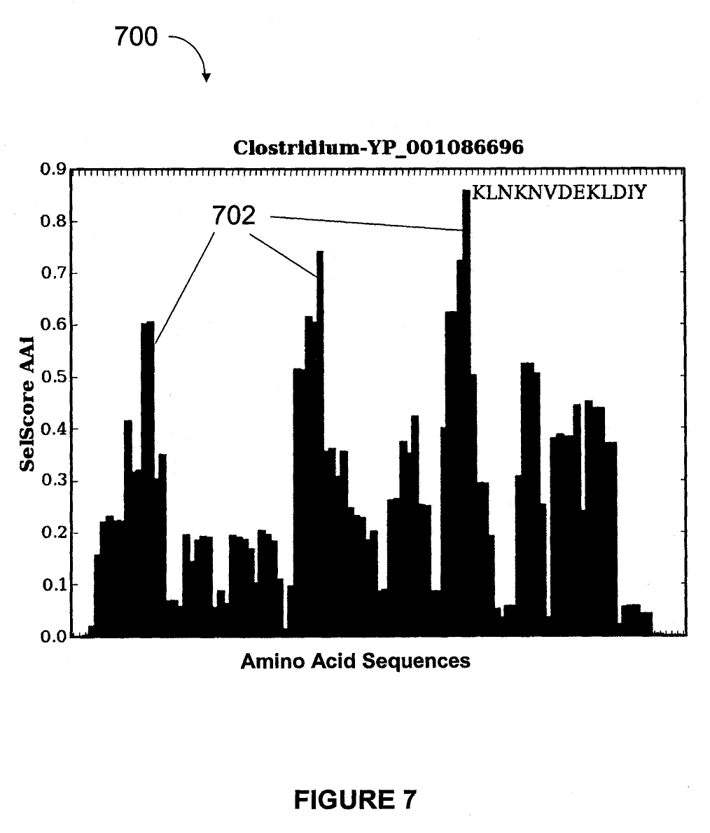

In step 328, a genome selection score database is output. In one exemplary embodiment, the amino acid sequence selection score, the protein selection score, and the genome selection score are stored to data storage device 106. In another exemplary embodiment, data processor 104 transmits electronic data to output device 108. The electronic data may include the amino acid sequence selection score, the protein selection score, and the genome selection score. Output device 108 may then present the selection scores to a user via, for example, a chart or graph indicating the comparative height of the selection scores for the one or more structurally or functionally significant amino acid sequences. FIG. 7 depicts an exemplary chart for depicting the selection scores for a set of amino acid sequences, as will be discussed below.

FIG. 4 is a flow chart of exemplary steps for outputting genome word dictionaries (step 314; FIG. 3) in accordance with an aspect of the present invention.

In step 402, a distance between the observed and expected frequencies of each amino acid word is calculated. In an exemplary embodiment, data processor 104 compares the observed frequency for each amino acid word in each protein encoded by the genome with the expected frequency for each amino acid word in each protein encoded by the genome. Data processor 104 may utilize a standard Euclidean distance calculation in order to plot a point in a two-dimensional space corresponding to the observed and expected frequencies of an amino acid word. The two dimensions may be the observed frequency and the expected frequency for amino acid words, with each plotted point corresponding to those frequencies for an amino acid word. The two dimensions may vary linearly or logarithmically. Data processor 104 may then compute a linear distance between the plotted point and a hypothetical 1:1 reference line in the two-dimensional space. The 1:1 reference line may correspond to points on the graph where the observed frequency is equal to the expected frequency for an amino acid word. The calculated distance may be the perpendicular distance between the observed vs. expected frequency point for an amino acid word and the 1:1 reference line, and may be calculated using Euclidean geometry.

In an alternative exemplary embodiment, data processor 104 may calculate a distance between the observed and expected frequencies for each amino acid word by determining the difference between the two frequencies through subtraction. The calculated distance between the observed and expected frequencies may be stored in data storage device 106.

In step 404, an amino acid word dictionary is compiled for each genome. In an exemplary embodiment, data processor 104 compiles an amino acid word dictionary for each amino acid word in each protein sequence encoded by the genome. The is amino acid word dictionary may include an entry for each amino acid word in each protein sequence encoded by the genome. Each entry may include the observed frequency, expected frequency, and calculated distance between the two frequencies for the amino acid word.

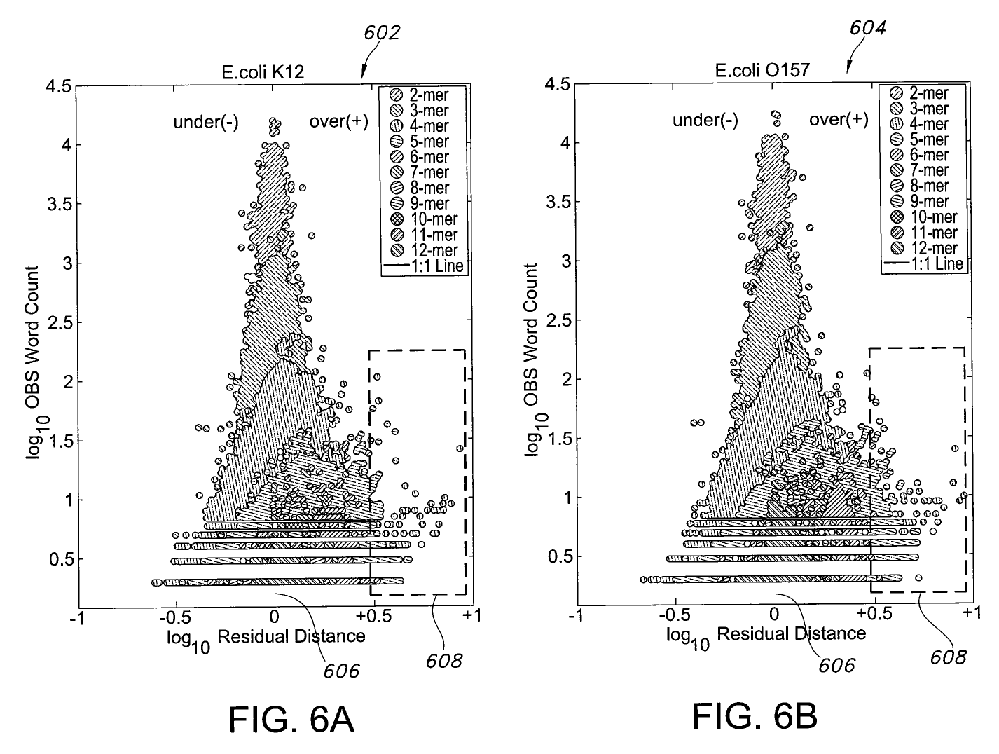

In step 406, the amino acid word dictionary for each genome is stored and/or presented. In one exemplary embodiment, the amino acid word dictionary for each genome may be stored in data storage device 106. In another exemplary embodiment, data processor 104 may transmit electronic data to output device(s) 108. The electronic data may include the amino acid word dictionary for each genome. Output device(s) 108 may then present amino acid word dictionary to a user by, for example, a chart or graph depicting the calculated distance between observed and expected frequencies for each amino acid word in each protein sequence encoded by a genome presented on a monitor or printed on paper. Electronic data transmitted to output device(s) 108 may be at least temporarily stored, e.g., in a video buffer (not shown). FIG. 6, described below, depicts an exemplary graph for depicting the calculated distance between observed and expected frequencies for each amino acid word in each protein sequence encoded by a genome, as will be discussed below.

FIG. 5 is an illustration 500 for use in explaining the determination of an amino acid sequence selection score for an amino acid sequence as described in step 322 of flow chart 300, in accordance with an aspect of the present invention. Illustration 500 depicts 12 amino acids (amino acids 502a-502l), five amino acid words (amino acid words 504a-504e), and one amino acid sequence (amino acid sequence 506). Additional details for determining a selection score are provided below.

The selection score for an amino acid sequence in a protein sequence may be determined based on the selection score for each amino acid in the sequence. Illustration 500 depicts a sample sequence of amino acids 502a-502l in a protein sequence. In an exemplary embodiment, data processor 104 examines every 4-long, 5-long, and 6-long amino acid word in each protein sequence. Example 500 depicts a series of 4-long amino acid words 504a-504e. For example, amino acid word 504a includes amino acids 502a-502d; amino acid word 504b includes amino acids 502b-502e; and so on.

Each amino acid word 504a-504e has a corresponding calculated distance between the word's observed and expected frequency, as contained in the amino acid is word dictionary generated in step 314. For each examined word 504a-504e, the calculated distance for the amino acid word is added to each amino acid in the amino acid word to generate a selection score for each amino acid. For example, assume amino acid word 504a has a calculated distance of 5; word 504b has a calculated distance of 6; word 504c has a calculated distance of 4; word 504d has a calculated distance of 6; and word 504e has a calculated distance of 7. In this example, the selection score for amino acid 502d would be the sum of the calculated distances for amino acid words 504a-504d, or 21 (5+6+4+6); the selection score for amino acid 502e would be the sum of the calculated distances for amino acid words 504b-504e, or 23 (6+4+6+7).

In an exemplary embodiment, data processor 104 performs this summation for each amino acid in the protein sequence using all 4-long amino acid words (e.g. 504a-504e), 5-long amino acid words (not shown), and 6-long amino acid words (not shown). Data processor 104 may then examine all 13-long amino acid sequences in the protein. Data processor 104 may determine a selection score for each 13-long amino acid sequence in each protein sequence encoded by the genome by summing the selection scores for each amino acid contained in the amino acid sequence. For example, the selection score for 13-long amino acid sequence 506 would be the sum of the selection scores for amino acids 502a-502k. Data processor 104 may store the selection score for the amino acid sequence in data storage device 106.

FIGS. 6A & 6B depict graphs 602 & 604, which show the calculated distance between observed and expected amino acid word frequencies for two genomes in accordance with an aspect of the present invention. Graph 602 corresponds to the amino acid word dictionary for the common non-pathogenic bacteria E. coli str. K12, and graph 604 corresponds to the amino acid word dictionary for the human pathogenic bacteria E. coli str. O157. Each graph includes a multitude of data points each corresponding to an amino acid word occurring in the protein sequences encoded by the genome of the corresponding bacteria.

Each graph further includes a line 606 corresponding to points where the observed and expected frequencies of each amino acid word in the protein sequences encoded by the genome are equal. For example, points falling to the right of line 606 correspond to amino acid words having an observed frequency greater than their expected frequency; points falling to the left of line 606 correspond to amino acid words having an observed frequency less than their expected frequency.

Region 608 on both graphs represents an exemplary location on each graph where amino acid words having substantially higher observed frequencies than would be expected. Amino acid sequences containing the amino acid words falling within region 608 may be sequences having high selection scores, as described above. Accordingly, amino acid sequences containing amino acid words falling within region 608 of graph 602 may be structurally or functionally significant to E. coli str. K12 bacteria, and amino acid sequences containing amino acid words falling within region 608 of graph 604 may be structurally or functionally significant to E. coli str. O157 bacteria.

Further, comparison of graphs 602 and 604 may demonstrate the differences in the genomes of non-pathogenic E. coli str. K12 and pathogenic E. coli str. O157. For example, if an amino acid word falls within region 608 of graph 604, but not within region 608 of graph 602, this may indicate that amino acid sequences containing the amino acid word are structurally or functionally significant to the pathogenic bacteria but not to the non-pathogenic bacteria. This comparison may provide further information on the different effects of natural selection on the genome of a pathogen as opposed to the effects of natural selection on the genome of a non-pathogen.

FIG. 7 depicts an exemplary chart 700 showing the selection scores for amino acid sequences in a protein sequence encoded by a genome in accordance with an aspect of the present invention. Specifically, chart 700 depicts the b 13-long amino acid sequence selection scores for the protein sequence YP-001086696, encoded by the genome of Clostridium difficile str. 630. Peaks 702 correspond to 13-long amino acid sequences having high selection scores as compared with the rest of the amino acid sequences, as calculated above. The highest amino acid sequence selection score in the protein sequence corresponds to the 13-long amino acid sequence “KLNKNVDEKLDIY.” Accordingly, this amino acid sequence is likely to be structurally or functionally significant to the protein sequence, and may be a good structure for antibiotic drug targeting, as described above.

One or more of the steps described above may be embodied in computer-executable instructions stored on a computer readable storage medium. The computer readable storage medium may be essentially any tangible storage medium capable of storing instructions for performance by a general or specific purpose computer such as an optical disc, magnetic disk, or solid state device, for example.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A computer implemented method for identifying at least one significant amino acid sequence encoded by a genome, comprising the steps of:
compiling an observed frequency for each of a plurality of amino acid words encoded by the genome;
calculating with a computer an expected frequency for each of the plurality of amino acid words encoded by the genome; and
identifying at least one significant amino acid sequence encoded by the genome based at least in part on the observed and expected frequencies for each of the plurality of amino acid words encoded by the genome.

2. The method of claim 1, wherein the step of identifying at least one significant amino acid sequence comprises:
determining a selection score for at least one amino acid sequence encoded by the genome based at least in part on the difference between the observed and expected frequencies for each of the plurality of amino acid words encoded by the genome, the selection score corresponding to the structural significance of the at least one amino acid sequence; and
identifying at least one significant amino acid sequence based on the selection score for the amino acid sequence.

3. The method of claim 1, wherein the step of calculating with a computer an expected frequency comprises:
calculating with a computer an expected frequency for each of the plurality of amino acid words encoded by the genome based at least in part on the observed frequency for at least one of the plurality of amino acid words encoded by the genome.

4. The method of claim 1, wherein the step of calculating with a computer an expected number of occurrences comprises:
calculating with a computer an expected frequency for each of the plurality of amino acid words encoded by the genome based at least in part on the observed frequencies of two or more amino acid subwords occurring within each of the plurality of amino acid words encoded by the genome.

5. The method of claim 1, wherein the plurality of amino acid words comprises amino acid words having from one to twelve amino acids.

6. The method of claim 1, wherein the at least one significant amino acid sequence comprises at least one significant amino acid sequence having thirteen amino acids.

7. The method of claim 2, further comprising the step of:
compiling selection scores for each amino acid sequence encoded by the genome.

8. The method of claim 7, further comprising the step of:
calculating a protein selection score for at least one protein sequence encoded by the genome based on the selection scores for each amino acid sequence occurring within the at least one protein sequence.

9. The method of claim 8, further comprising the step of:
calculating a genome selection score for the genome based on the selection scores for each protein sequence encoded by the genome.

10. The method of claim 1, wherein the step of calculating with a computer an expected frequency comprises:
transforming with a computer the observed frequency for each of the plurality of amino acid words encoded by the genome into an expected frequency for each of the plurality of amino acid words encoded by the genome.

11. The method of claim 1, wherein the step of identifying the at least one significant amino acid sequence comprises:
transforming the observed and expected frequencies for each of the plurality of amino acid words encoded by the genome into a selection score for at least one amino acid sequence encoded by the genome, the selection score corresponding to the structural significance of the at least one amino acid sequence.

12. The method of claim 1, wherein the step of identifying the at least one significant amino acid sequence comprises:
identifying the at least one significant amino acid sequence encoded by the genome based at least in part on the observed and expected frequencies for each of the plurality of amino acid words encoded by the genome and observed frequency differences between at least one of the plurality of amino acid words encoded by the genome and encoded by a related genome.

13. The method of claim 12, wherein the genome is a pathogenic genome and the related genome is a non-pathogenic genome.

14. The method of claim 1, wherein the at least one significant amino acid sequence comprises at least one structurally significant amino acid sequence.

15. The method of claim 1, wherein the at least one significant amino acid sequence comprises at least one functionally significant amino acid sequence.

16. A method for targeting at least one significant amino acid sequence in the protein of a pathogen, comprising the steps of:
compiling an observed frequency for each of a plurality of amino acid words encoded by the genome of the pathogen;
calculating with a computer an expected frequency for each of the plurality of amino acid words encoded by the genome of the pathogen;
identifying at least one significant amino acid sequence encoded by the genome of the pathogen based at least in part on the observed and expected frequencies for each of the plurality of amino acid words encoded by the genome of the pathogen; and
developing a drug configured to interact with the at least one significant amino acid sequence encoded by the genome of the pathogen.

17. The method of claim 16, wherein the step of identifying at least one significant amino acid sequence comprises
determining a selection score for at least one amino acid sequence encoded by the genome based at least in part on the difference between the observed and expected frequencies for each of the plurality of amino acid words encoded by the genome, the selection score corresponding to the structural significance of the at least one amino acid sequence; and
identifying at least one significant amino acid sequence based on the selection score for the amino acid sequence.

18. The method of claim 17, wherein the step of developing a drug comprises:
developing a drug configured to interact with the at least one significant amino acid sequence encoded by the genome of the pathogen based at least in part on the selection score for the at least one significant amino acid sequence encoded by the genome of the pathogen.

19. The method of claim 17, wherein the step of developing a drug comprises:
developing a drug configured to interact with the at least one significant amino acid sequence encoded by the genome of the pathogen based at least in part on another selection score for the at least one significant amino acid sequence encoded by another genome.

20. The method of claim 16, wherein the at least one significant amino acid sequence comprises at least one structurally significant amino acid sequence.

21. The method of claim 16, wherein the at least one significant amino acid sequence comprises at least one functionally significant amino acid sequence.

22. The method of claim 16, wherein the step of identifying the at least one significant amino acid sequence comprises:
identifying the at least one significant amino acid sequence encoded by the genome based at least in part on the observed and expected frequencies for each of the plurality of amino acid words encoded by the genome and observed frequency differences between at least one of the plurality of amino acid words encoded by the genome and encoded by a related genome.

23. The method of claim 22, wherein the related genome is a non-pathogenic genome.

24. A system for identifying at least one significant amino acid sequence in a genome, the system comprising:
means for compiling an observed frequency for each of a plurality of amino acid words encoded by the genome;
means for calculating with a computer an expected frequency for each of the plurality of amino acid words encoded by the genome; and
means for identifying at least one significant amino acid sequence encoded by the genome based at least in part on the observed and expected frequencies for each of the plurality of amino acid words encoded by the genome.

25. The system of claim 24, wherein the identifying means comprises:
means for identifying the at least one significant amino acid sequence encoded by the genome based at least in part on the observed and expected frequencies for each of the plurality of amino acid words encoded by the genome and observed frequency differences between at least one of the plurality of amino acid words encoded by the genome and encoded by a related genome.

26. A computer-readable medium encoded with instructions for execution by a computer to implement a method for identifying at least one significant amino acid in a genome, the method comprising the steps of:
compiling an observed frequency for each of a plurality of amino acid words encoded by the genome;
calculating an expected frequency for each of the plurality of amino acid words encoded by the genome; and
identifying at least one significant amino acid sequence encoded by the genome from the observed and expected frequencies for each of the plurality of amino acid sequences encoded by the genome.

27. The computer-readable medium of claim 26, wherein the step of identifying the at least one significant amino acid sequence comprises:
identifying the at least one significant amino acid sequence encoded by the genome based at least in part on the observed and expected frequencies for each of the plurality of amino acid words encoded by the genome and observed frequency differences between at least one of the plurality of amino acid words encoded by the genome and encoded by a related genome.
